(19) United States
(12) Patent Application Publication
(10) Pub. No.: US 2026/0207340 A1
(43) Pub. Date: Jul. 23, 2026

(54) RADIAL HEAD SURGICAL IMPLANT

(71) Applicant: BIOPOLY, LLC, Fort Wayne, IN (US)

(72) Inventors: Herbert E. SCHWARTZ, Fort Wayne, IN (US); Jacob ALUMBAUGH, Fort Wayne, IN (US)

(73) Assignee: BIOPOLY, LLC, Fort Wayne, IN (US)

(21) Appl. No.: 19/570,738

(22) Filed: Mar. 18, 2026

Related U.S. Application Data
(63) Continuation of application No. PCT/US2024/047254, filed on Sep. 18, 2024.
(60) Provisional application No. 63/583,744, filed on Sep. 19, 2023.

(51) Int. Cl. A61F 2/38; A61F 2/30

(52) U.S. Cl. CPC A61F 2/3804; A61F 2002/30011; A61F 2002/30354; A61F 2002/30387; A61F 2002/3093; A61F 2002/3809; A61F 2002/3827

(57) ABSTRACT

A radial head replacement implant includes a polymer head and a biocompatible metal stem component. The polymer head includes an articulating surface and an outer head perimeter. The stem component includes a stem and a mounting platform. The stem is configured to be inserted into a bone of a patient. The mounting platform is configured to have the polymer head attached thereon. The mounting platform has a mounting platform outer perimeter. The outer head perimeter of the polymer head is larger than the mounting platform outer perimeter. A first portion of the polymer head extends around the mounting platform outer perimeter and under the mounting platform to secure the polymer head to the mounting platform.

### CROSS REFERENCE TO RELATED APPLICATIONS

This application is a bypass continuation application under 35 U.S.C. § 111(a) of International Application PCT/US 2024/047254, filed Sep. 18, 2024, and published as WO 2025/064527 A1 on Mar. 27, 2025. PCT/US2024/047254 claims priority from U.S. provisional application 63/583744, filed Sep. 19, 2023. The entire contents of each of these prior applications are hereby incorporated herein by reference.

### TECHNICAL FIELD

The present disclosure relates to an orthopedic implant for the replacement of the distal radial head in an upper extremity. More specifically, but not exclusively, the present disclosure relates to a modular radial head implant that utilizes a combination of a metal substrate molded with a polymer articulating surface.

### BACKGROUND OF THE INVENTION

Many currently available surgical implants for the replacement of the radial head do not completely address the needs of patients and fail to account for the properties of joint anatomy and cartilage degradation and accordingly can decrease favorability of the outcome for the patient.

For example, the distance between a resected distal end of a bone, such as a damaged radius bone, and the bone it normally articulates with, such as the humerus, may vary depending on the size of a patient and the extent of injuries to the radius. Additionally, an all metal implant may not react favorably with the remaining articular cartilage and are known to degrade cartilage. If a polymer were used as the articulating component of the radial head implant, any polymer portion of an implant may prematurely wear or not be properly anchored to the metal portion of an implant.

Accordingly, there is a need for a radial head replacement implant that is constructed to reduce wear on any polymer portion of the implant and to reduce damage to the opposing cartilage with which it articulates. There is also a need for a radial head replacement implant, or implant kit, that can compensate for a variable space between the distal end of a resected damaged bone and the bone that the damaged bone normally articulates with.

### SUMMARY OF THE INVENTION

The present disclosure offers advantages and alternatives over the prior art by providing a radial head replacement implant that includes a polymer head, a stem component and one or more spacers. The spacer can provide an adjustable offset between the polymer head of the implant and the resected distal end of the bone the implant is implanted into. The implant also includes a mounting platform on the stem component upon which the polymer head is molded, which has a geometry that enables a more secure anchoring of the polymer head to the mounting platform. The implant also reduces wear of the polymer head by eliminating any contact between the bone the implant is implanted into and the space that contacts the mounting platform.

A radial head replacement implant in accordance with one or more aspects of the present disclosure includes a polymer head and a biocompatible metal stem component. The polymer head includes an articulating surface and an outer head perimeter. The stem component includes a stem and a mounting platform. The stem is configured to be inserted into a bone of a patient. The mounting platform is configured to have the polymer head attached thereon. The mounting platform has a mounting platform outer perimeter. The outer head perimeter of the polymer head is larger than the mounting platform outer perimeter. A first portion of the polymer head extends around the mounting platform outer perimeter and under the mounting platform to secure the polymer head to the mounting platform.

Another radial head replacement implant in accordance with one or more aspects of the present disclosure includes a polymer head, a biocompatible metal stem component and a spacer. The polymer head includes an articulating surface and an outer head perimeter. The stem component includes a stem and a mounting platform. The stem is configured to be implanted into a bone of a patient. The mounting platform is configured to have the polymer head attached thereon. The mounting platform has a mounting platform outer perimeter. The spacer is configured to be disposed on the stem and configured to contact the mounting platform. The spacer includes a spacer diameter that is configured smaller than the mounting platform outer perimeter and large enough to abut against an outer perimeter of the bone that the implant is implanted into. The spacer also includes a spacer height that is configured to provide a predetermined offset of the polymer head from the bone the implant is implanted into.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits and advantages described herein.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

The disclosure will be more fully understood from the following detailed description taken in conjunction with the accompanying drawings, in which:

FIG. 1 is a front top perspective view of a radial head implant having a polymer head and a stem component, in accordance with the present disclosure;

FIG. 2 is a bottom perspective view of the implant of FIG. 1, in accordance with the present disclosure;

FIG. 3A is a side view of the stem component of the implant of FIG. 1, in accordance with the present disclosure;

FIG. 3B is a top view of the stem component of the implant of FIG. 1, in accordance with the present disclosure;

FIG. 3C is an enlarged view of circular area A of the implant of FIG. 1, in accordance with the present disclosure;

FIG. 4 is a side view of the implant of FIG. 1 showing the stem component structure in dotted line format hidden within the polymer head, in accordance with the present disclosure;

FIG. 5 is a perspective view of the implant of FIG. 1 implanted into a resected radius bone, in accordance with the present disclosure;

FIG. 6A is a side view of the implant of FIG. 1 with no spacer, in accordance with the present disclosure;

FIG. 6B is a side view of the implant of FIG. 1 with a spacer having a first spacer height, in accordance with the present disclosure;

FIG. 6C is a side view of the implant of FIG. 1 with a spacer having a second spacer height which is larger than the first spacer height, in accordance with the present disclosure;

FIG. 7 is a side view of the implant of FIG. 6A, with the polymer head partially cutaway, in accordance with the present disclosure;

FIG. 8 is a front top perspective view of a second embodiment of a radial head implant, in accordance with the present disclosure;

FIG. 9 is a bottom perspective view of the implant FIG. 8, showing multiple spacers, in accordance with the present disclosure;

FIG. 10 is a front top perspective view of a third embodiment of a radial head implant, in accordance with the present disclosure;

FIG. 11 is a bottom perspective view of the implant FIG. 10 showing a plurality of spacers, in accordance with the present disclosure;

FIG. 12 is a front top perspective view of a fourth embodiment of a radial head implant, in accordance with the present disclosure;

FIG. 13 is a bottom perspective view of the implant FIG. 12, in accordance with the present disclosure;

FIG. 14 is a side elevational view of the implant of FIG. 12, in accordance with the present disclosure;

FIG. 15 is a front top perspective view of a fifth embodiment of a radial head implant, in accordance with the present disclosure;

FIG. 16 is a bottom perspective view of the implant FIG. 15, in accordance with the present disclosure; and

FIG. 17 is a side elevational view of the implant of FIG. 15 in accordance with the present disclosure;

### DETAILED DESCRIPTION OF THE INVENTION

Certain examples will now be described to provide an overall understanding of the principles of the structure, function, manufacture, and use of the methods, systems, and devices disclosed herein. One or more examples are illustrated in the accompanying drawings. Those skilled in the art will understand that the methods, systems, and devices specifically described herein and illustrated in the accompanying drawings are non-limiting examples and that the scope of the present disclosure is defined solely by the claims. The features illustrated or described in connection with one example may be combined with the features of other examples. Such modifications and variations are intended to be included within the scope of the present disclosure.

The terms “significantly”, “substantially”, “approximately”, “about”, “relatively,” or other such similar terms that may be used throughout this disclosure, including the claims, are used to describe and account for small fluctuations, such as due to variations in processing from a reference or parameter. Such small fluctuations include a zero fluctuation from the reference or parameter as well. For example, they can refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features.

Furthermore, the implant or structure that is configured in a certain way is configured in at least that way, but may also be configured in ways that are not listed.

The present disclosure is directed toward a stemmed radial head surgical implant for the replacement of the diseased distal radial head.

The radial head surgical implant includes an articulating polymer head, which may be made of a combination of Hyaluronic Acid and Ultra High Molecular Weight Polyethylene (UHMWPE). The polymer head interacts favorably with the remaining articular cartilage. The polymer head would replace the metal utilized in other commercially available traditional radial head replacement implants.

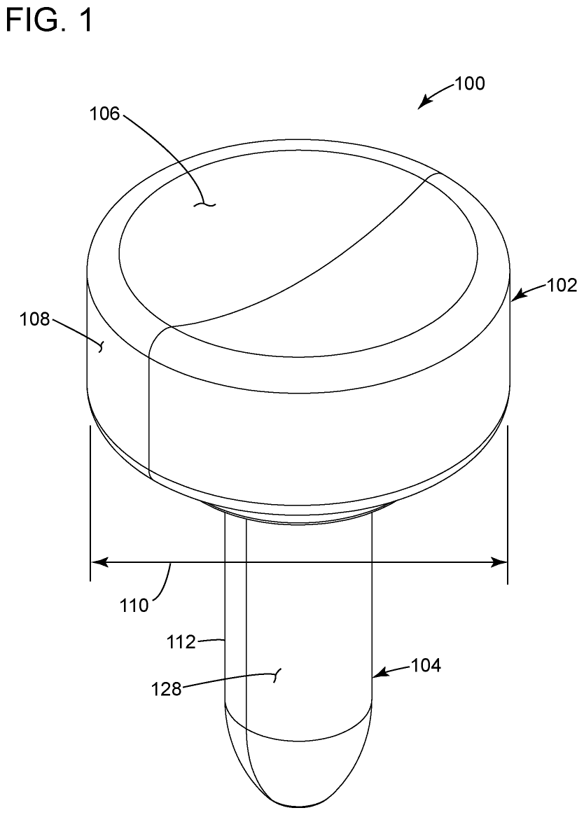

The radial head implant includes a polymer head and a stem component. The polymer head, for example, has an outer shape that may be generally cylindrical, although other shapes are contemplated (i.e., oval, square orthogonal, spherical, etc.) and has a generally concave articular surface with rounded edges. (See FIGS. 1, 8, 10, 12 and 15). The polymer head shape may be symmetrical, designed as a spacer in the joint, or asymmetrical, as an anatomic replacement for the radial head geometry. The polymer head may have multiple diameters to accommodate various patient anatomy with the various sized implants being available in a kit.

The polymer head may be comprised of all polymer with the concave articulating surface being positioned on the top aspect of the polymer head. An additional embodiment of the polymer head may be a polymer articulating member attached onto a metal backed member. By way of a non-limiting example, the polymer head may be molded onto the metal backed member.

The stem component may be fabricated from a biocompatible metal. Examples of such materials include titanium alloy, commercially pure titanium, cobalt chrome alloy, or stainless steel. The outer surface of the stem component may be smoothly polished and undersized to allow for free rotation in the radial canal. Multiple diameters of the stem component are contemplated to accommodate the varying patient population anatomy with the various sizes being available in a kit.

Alternatively, the stem component may be fabricated with an oversized porous on-growth surface or a nanosurface, to fix the implant in the radial canal. In addition, the stem may have various bone growth materials (i.e., HA, TCP) applied to either the smooth or porous surface to facilitate in-growth of bone.

The stem component may also be configured with different height offsets for the head component to accommodate proper joint spacing. Alternatively, these height offsets may be provided by polymer heads having different overall heights or lengthening features. The height offsets may also be provided by spacers have various heights.

Various stem designs of the stem component are contemplated, including stems that have rounded distal tips, blunt or square distal tips, tapered distal tips and/or pointed distal tips. The outer surface of the stems may be smooth or uninterrupted or may include channels positioned longitudinally along the full and or partial length of the stem. The various stem designs contemplated are to facilitate bone fixation post-implantation of the radial head implant. The various sized and shaped stems may be available with different bone enhancement surface coatings as noted above.

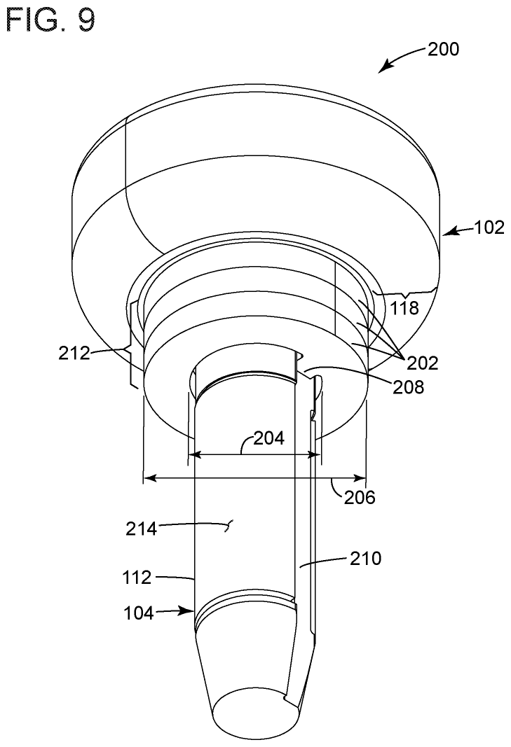

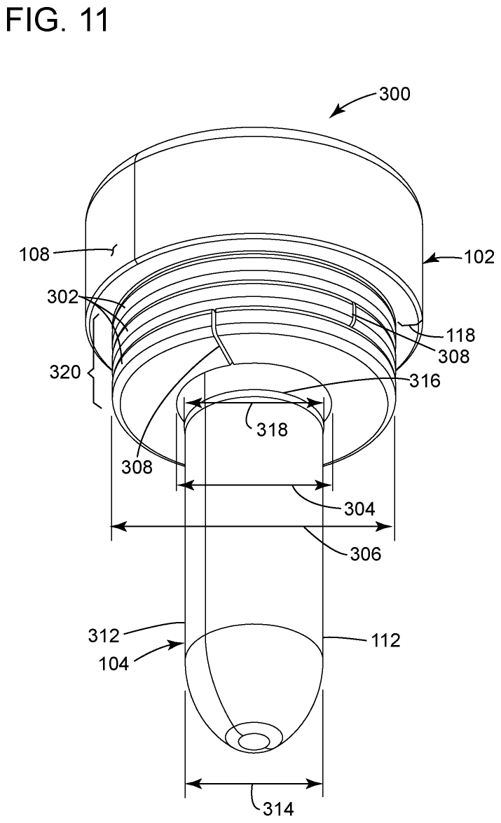

The height offset may be accomplished by modular spacers, or spacing rings, which can be slid, tapped or pressed onto the stem of the stem component proximal to the undersurface of the polymer head (See FIGS. 9 and 11) to provide height offset. The modular spacers may also loosely fit onto the stem, if the stem is fixed.

The radial head implant may be monolithic in construct with the stem being integral to the head component. A kit may include several single piece radial head implants that have various sized polymer head thicknesses, stem lengths and stem diameters and height offset sizes to allow the surgeon to choose the best fit implant for a patient on which the surgery is being performed.

The radial head implant may also be fabricated as a modular design. The surgeon may be able to assemble the implant by attaching the stem component to the under surface of the polymer head. The stem component may be attached to the polymer head in possible combination with the various height-modifying spacers, or spacer rings. The polymer articulating head-stem component attachment mechanism may be for example a press fit design, a threaded connection or a barbed configuration. The arrangement mechanism may allow for fixation directly into the polymer material of the head component. The polymer material of the head may also be fit into a lip on the stem, similar to tibial trays for total knee arthroplasty implants. The polymer head may also contain a magnet element that provides a connection to another magnet in the stem component. The polymer head may also be over-molded on a metal portion for connection to the stem component. In the event the polymer head is comprised of a polymer articulating surface member molded to a metal backed member, the modular stem component may then be attached to the metal backed member using various mechanical mechanisms, for example a morse taper, a threaded male/female arrangement or other similar modular connector arrangements.

The design of the radial head implant is for single use and may be available in over 30 different configurations of articulating polymer head diameters, polymer head heights, stem component lengths, stem component diameters and spacer, or spacer ring, thicknesses. All of these different configurations may be assembled individually or as a kit.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features.

Furthermore, the implant or structure that is configured in a certain way is configured in at least that way, but may also be configured in ways that are not listed.

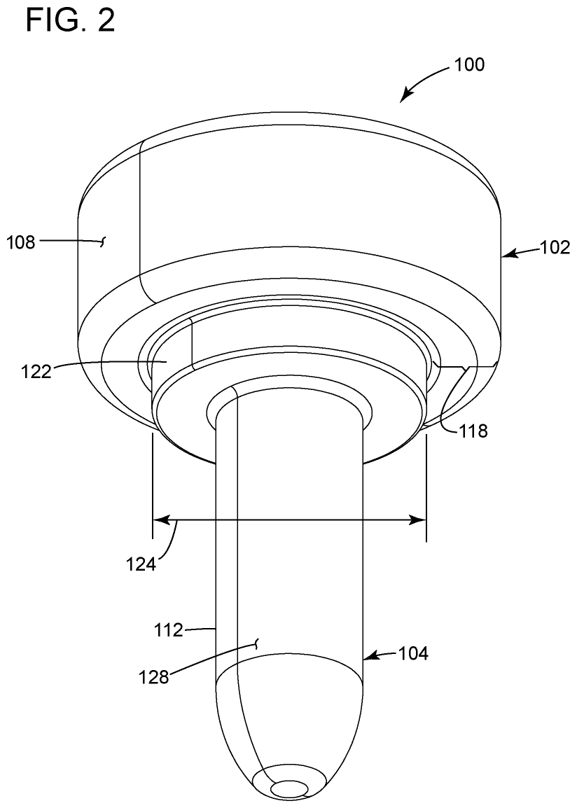

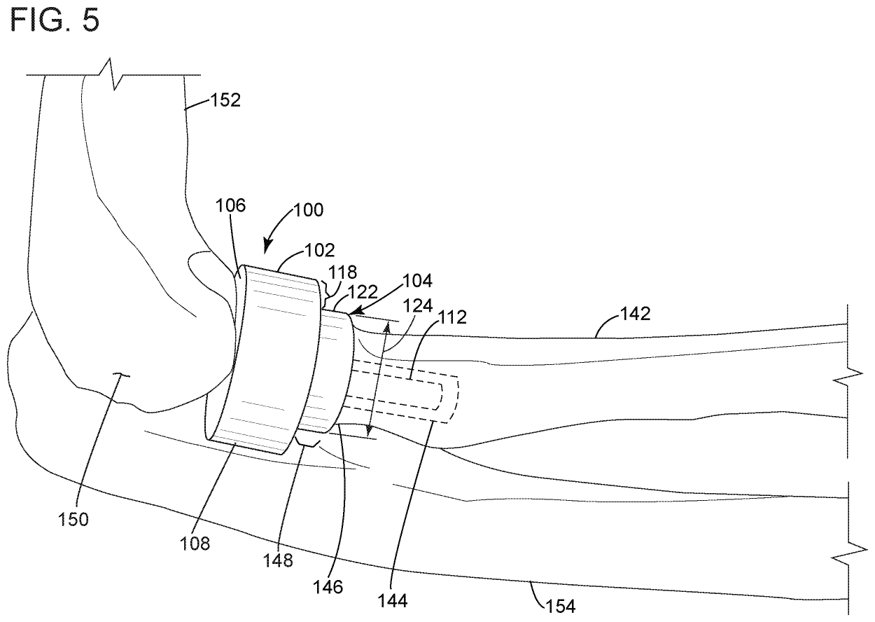

Referring to FIGS. 1 and 2, depict an example of a front top perspective view (FIG. 1) and a bottom perspective view (FIG. 2) of a radial head implant 100 having a polymer head 102 and a stem component 104, in accordance with the present disclosure. As will be explained in greater detail herein, the implant 100 is designed to be implanted into a resected distal end of a bone, such as a damaged radius bone 142 (see FIG. 5) in an elbow joint.

More specifically, the radial head replacement implant 100 includes a polymer head 102 and a biocompatible stem component 104. The polymer head may be composed of, for example, a combination of Hyaluronic Acid and UHMWPE. The biocompatible metal stem component may be composed of, for example, such materials as titanium alloy, commercially pure titanium, cobalt chrome alloy, stainless steel.

The polymer head 102 includes an articulating surface 106 and an outer head perimeter 108. The articulating surface 106 is concave in shape and designed to articulate with bones in a joint that the implant 100 is designed to repair. By way of example, the implant 100 may be implanted into a resected distal end of a radius in a damaged elbow joint (see FIG. 5), wherein the concave articulating surface 106 may articulate with the capitellum 150 of the humerus bone 152. The outer head perimeter 108, in this case, is circular and has a diameter 110. In the above example, the outer head perimeter 108 of the polymer head 102 may articulate with the ulna bone 154.

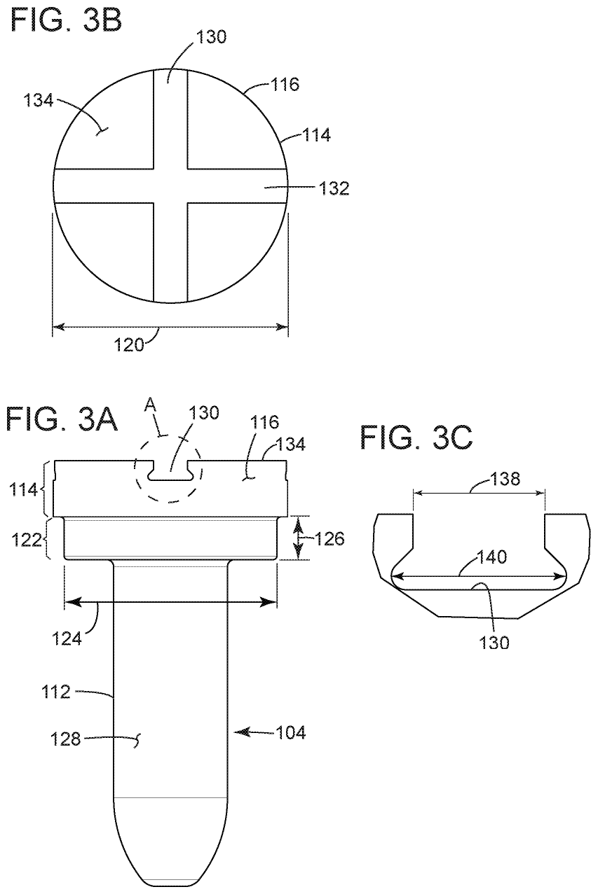

The stem component includes a stem (or stem portion) 112 and a mounting platform 114 (see FIG. 3A). The stem 112 is configured to be implanted into a bone (for example a radius bone) of a patient. The mounting platform 114 is configured to have the polymer head 102 attached thereon. The mounting platform 114 includes a mounting platform outer perimeter 116 (see FIG. 3A), which, in the examples provided in FIGS. 1-17, is circular in shape and has an overall diameter 120 (see FIG. 3B).

By way of a non-limiting example, the polymer head 102 may be attached to the mounting platform 114 by molding the polymer head 102 thereon. Alternatively, various attachment mechanisms or fasteners may be used to attach the polymer head 102 to the mounting platform 114.

As will be explained in greater detail herein, the outer head perimeter 108 of the polymer head 102 is larger than the mounting platform outer perimeter 116. Moreover, a first portion 118 of the polymer head 102 extends around the mounting platform outer perimeter 116 and under the mounting platform 114 to firmly secure the polymer head 102 to the mounting platform 114 of the stem component 104.

The implant 100 may include a spacer 122, which is configured to be disposed on the stem 112 and configured to contact the mounting platform 114. The spacer 122 includes a spacer diameter and a spacer height 126 (see FIG. 3A). The spacer diameter 124 is sized smaller than the mounting platform outer perimeter 116, and, therefore, is smaller than the overall diameter 120 of the mounting platform 114. Moreover, the spacer diameter 124 is sized small enough such that the spacer 122 does not overlap the first portion 118 of the polymer head 102 that extends under the mounting platform 114. However, the spacer diameter 124 is also sized large enough to abut against an outer perimeter 146 of the bone 142 that the implant 100 is implanted into (see FIG. 5). The spacer height 126 that is configured to provide a predetermined offset 148 of the polymer head 102 from the bone 142 that the implant 100 is implanted into (see FIG. 5) In the examples provide in FIGS. 1-7, the spacer 122 of implant 100 is integrally connected to the mounting platform 114 and the stem 112 of the stem component 104. Therefore, in implant 100, the stem component 104 and spacer 122 are a single piece structure. Also in the examples provided in FIGS. 1-7, the stem includes a smooth circular outer surface 128 that is configured to enable the implant 100 to spin within the bone 142 the implant 100 is implanted into.

Referring to FIGS. 3A-3C, an example is depicted of a side view (FIG. 3A) and a top view (FIG. 3B) of the stem component 104 of the implant 100; and an enlarged view of circular area A (FIG. 3C), which shows a first dovetail groove 130 disposed in the stem component 104.

As discussed earlier, the stem component 104 includes the stem 112 and mounting platform. The spacer 122 is configured to be disposed on the stem 112 and contact the mounting platform 114. The spacer 122 may be integrally connected to the stem component 104 as is the case with implant 100, or may be a separate modular piece that can be assembled onto the stem 112 during a surgical procedure. Moreover, the spacer's diameter 124 is configured to be smaller than the mounting platform outer perimeter 116 such that the spacer 122 does not overlap the first portion 118 of the polymer head 102 that extends under the mounting platform 114 and large enough to abut against an outer perimeter of the bone 142 that the implant 100 is implanted into. The spacer height 126 is configured to provide a predetermined offset 148 of the polymer head 102 from the bone 142 the implant 100 is implanted into.

The first portion 118 of the polymer head 102 extending under the mounting plate 114 grips the underside of the mounting plate 114 to help prevent the polymer head 102 from separating from the stem component 102 during use. However additional support for securing the polymer head 102 to the stem component 104 may be provided with a first and a second dovetail groove 130, 132 (see FIG. 3C) that may be positioned in the upper surface 134 of the mounting platform 114.

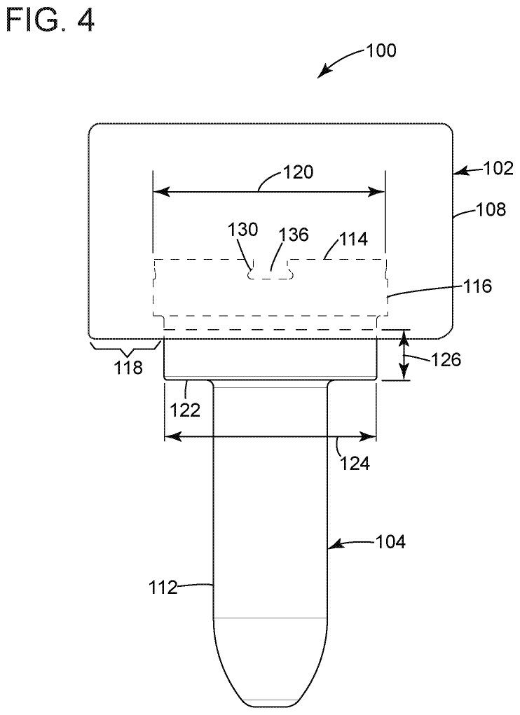

More specifically, the first dovetail groove 130 is disposed in the upper surface 134 of the mounting platform 114 and extends across the entire diameter 120 of the mounting platform 114. Accordingly, a second portion 136 (see FIG. 4) of the polymer head 102 extends into the first dovetail groove 130 to further secure the polymer head 102 to the mounting platform 114.

Further the second dovetail groove 132 is disposed in the upper surface 134 of the mounting platform 114 and extends perpendicularly relative to the first dovetail groove 130 across the entire diameter 120 of the mounting platform 114. Accordingly, a third portion (not shown) of the polymer head 102 extends into the second dovetail groove 132 to further secure the polymer head 102 to the mounting platform 114.

Referring more specifically to FIG. 3C, the first dovetail groove 130 is shown.

However, the second dovetail groove 132 may be of similar structure. The first dovetail groove 130 tapers downwardly from a narrow opening 138 to a wider bottom 140. The second portion 136 of the polymer head 102 extrudes into the tapered wider bottom 140 to help lock the polymer head 102 in place on top of the mounting platform 114. The third portion (not shown) of the polymer head 102 is also locked into the second dovetail groove 132 in a similar fashion.

Referring to FIG. 4, an example is depicted of a side view of the implant 100 showing the stem component 104 structure in dotted line format hidden within the polymer head 102, in accordance with the present disclosure. As can be seen, the outer head perimeter 108 of the polymer head 102 is larger than the mounting platform outer perimeter 116 and, in this case, larger than the overall diameter 120 of the mounting platform 114. The first portion 118 of the polymer head 102 extends around the mounting platform outer perimeter 116 and under the mounting platform 114 to secure the polymer head 102 to the mounting platform 114 of the stem component 104.

Advantageously, the first portion 118 helps to lock the polymer head 102 to the stem component 104 and to prevent the polymer head 102 from separating from, or popping off of, the stem component 102. Additionally, the second portion 136 of the polymer head 102 extrudes into the first dovetail groove 130 to further secure the polymer head 102 to the stem component 104. Also, the third portion (not shown), of the polymer head 102 extrudes into the second dovetail groove 132 (see FIG. 3B) to further secure the polymer head 102 to the stem component 104.

The spacer 122, in this case, is integrally connected to the mounting platform 114 and the stem 112 of the stem component 104. Therefore, the stem component 104 and spacer 122 of implant 100 are structured as a single piece.

The spacer diameter 124 of the spacer 122 is sized smaller than the overall diameter 120 of the mounting platform 114 such that the spacer 122 does not overlap the first portion 118 of the polymer head 102 that extends under the mounting platform 114. In this case, the spacer 122 abuts the first portion 118 of the polymer head, but the spacer 122 does not overlap the polymer of the polymer head 102.

Referring to FIG. 5, an example is depicted of a perspective view of the implant 100 implanted into a resected radius bone 142, in accordance with the present disclosure. The stem 112 of the stem component 104 is disposed in a drilled out canal 144 of the radius 142. In this case, the diameter of the stem 112 is sized slightly smaller than the diameter of the canal 144 to allow the implant to spin.

The spacer diameter 124 is sized larger than an outer perimeter 146 of the resected distal end of the bone 142 the spacer 122 abuts against. Therefore the spacer 122 is large enough to abut against the entire outer perimeter 146 of the resected distal end of the radius bone 142.

The spacer height 122 (see FIG. 3A) is configured to provide a predetermined offset 148 of the polymer head 102 from the bone 142 that the implant 100 is implanted into. In this case, the spacer 122 provides an offset 148 between the polymer head 102 and the resected distal end of the radius bone 142.

Advantageously, the offset 148 ensures that the first portion 118 of the polymer head 102 that extends under the mounting platform 114 does not contact any bone. This prevents wear of the polymer of the first portion 118, which may cause the polymer head 102 to separate from the stem component 104 of the implant 100. Also advantageously, the offset 148 fills up the distance between the resected distal end of the radius bone 142 and the capitellum 150 of the humerus 152. The concave articulating surface 106 articulates with the capitellum 150 of the humerus 152. The outer head perimeter 108 articulates with the ulna bone 154.

The combination of the first portion 118 of the polymer head 102 extending under the mounting platform 114 to lock the stem component 104 to the polymer head 102, plus the spacer diameter 124 being sized larger than the outer perimeter 146 of the radius bone 142 to prevent wear on the first portion 118 provides a unique locking system of the polymer head 102 to the stem component 104. This unique locking system advantageously provides a more secure and long term locking system of the polymer head 102 to the stem component 104 over that of other prior art implants.

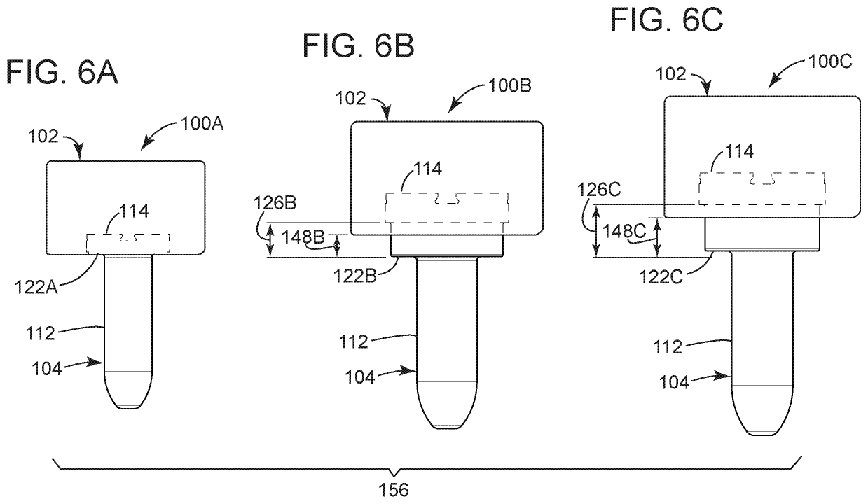

Referring to FIGS. 6A, 6B and 6C, an example is depicted of a plurality, or a kit 156, of implants 100 in accordance with the present disclosure. More specifically, FIG. 6A is a side view of a first implant 100A of the kit 156 of implants 100, with the special case of a zero height spacer 122A (or no spacer) with a spacer height 126 of zero. FIG. 6B is a side view of a second implant 100B of the kite 156 of implants 100, with a spacer 122B having a first spacer height 126B that is larger than zero. FIG. 6C is a side view of a third implant 100C of the kit 156 of implants 100, with a spacer 122C having a second spacer height 126C, wherein the second spacer height 126C is larger than the first spacer height 126B. Accordingly, each implant 100A, 100B, 100C, of the kit 156 of implants 100, has a spacer 122A, 122B, 122C (including the special case of a zero height spacer 122A with a spacer height of zero) that has a different spacer height 126.

Though the kit 156 of the implants 100 shows three implants 100A, 100B, 100C, such a kit 156 may include any number of implants 100 with any number of spacers 122 and any number of spacer heights 126. During a surgical procedure, a surgeon may choose the implant 100 that provides the optimum offset 148 between the polymer head 102 and the distal end of the bone, such as the radius bone 142, for the procedure. In the case of the three implants 100A, 100B, 100C of kit 156, the surgeon would have a choice of a zero offset by using implant 100A, a first non-zero offset 148A by using implant 100B or a second offset 148B that is that is larger than the first offset 148A by using implant 100C.

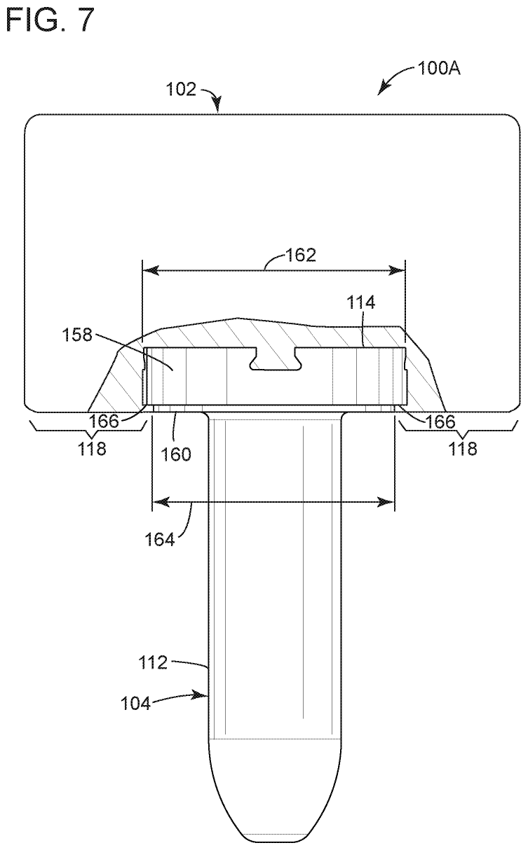

Referring to FIG. 7, an example is depicted of a side view of the implant 100A of FIG. 6A, with the polymer head 102 partially cut away, in accordance with the present disclosure. The implant 100A is a special case of no spacer, wherein the outer perimeter 146 of the resected bone 142 abuts against the bottom surface of the mounting platform 114. In order to preserve the advantage of a long term locking system, wherein the first portion 118 of the polymer head 102 extends under the mounting platform 114, plus the first portion 118 does not contact any bone that could cause wear, the mounting platform 114 is divided into an upper platform 158 and a lower platform 160.

More specifically, in the case of implant 100A, the mounting platform 114 includes a circular upper platform 158 having a first diameter 162 that forms the mounting platform outer perimeter 116 and a circular lower platform 160 having a second diameter 164 smaller than the first diameter 162. However, the second diameter 164 of the circular lower platform 160 is sized large enough to abut against an outer perimeter 146 of the bone 142 that the implant 100A is implanted into (see FIG. 5). In other words, the diameter 164 of the lower platform 160 is sized larger than the outer perimeter 146 of the resected distal end of the bone 142 the bottom platform 160 abuts against.

Additionally, the upper platform 158 and lower platform 160 form a lip 166 disposed on an under-surface of the upper platform 158 between the first diameter 162 and second diameter 164. Additionally the first portion 118 of the polymer head 102 that extends under the mounting platform 114 is disposed under the lip 166 and is flush with an under-surface of the lower platform 160.

Accordingly, the first portion 118 of the polymer head 104 still extends under the mounting platform 114 (in this case, the first portion 118 extends under the lip 166 of the mounting platform) to secure the polymer head 102 to the stem component 104. Additionally, the first portion 118 does not contact any bone 142 that may cause wear, because the diameter 164 is advantageously larger than the outer perimeter 146 of the bone 142 that the lower platform 160 abuts against.

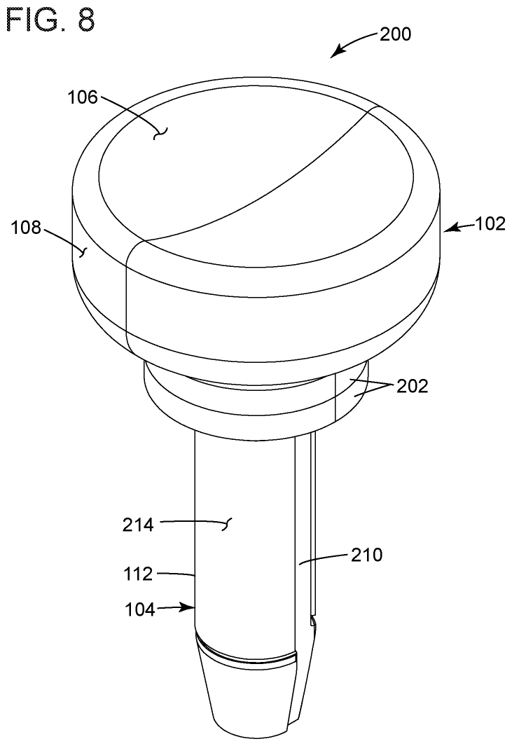

Referring to FIGS. 8 and 9, an example is depicted of a front top perspective view (FIG. 8) and a bottom perspective view (FIG. 9) of a second embodiment 200 of a radial head implant, in accordance with the present disclosure. The implant 200 is similar in many respects to that of implant 100 and, therefore, like or similar features are labeled with the same reference numbers.

Accordingly, implant 200 includes a polymer head 102 and a biocompatible metal stem component 104. The polymer head 102 includes an articulating surface 106 and an outer head perimeter 108. The stem component includes a stem 112 and a mounting platform 114 (see FIG. 3A). The stem 112 is configured to be implanted into a bone 142 of a patient. The mounting platform 114 is configured to have the polymer head 102 attached thereon. The outer head perimeter 108 of the polymer head 102 is larger than the mounting platform 114 outer perimeter 116. A first portion 118 of the polymer head 102 extends around the mounting platform outer perimeter 116 and under the mounting platform 114 to secure the polymer head 102 to the mounting platform 114 (see FIG. 4).

However, the spacers 202 of implant 200 are not integrally connected to the stem component 104. Rather the spacers 202 are configured as ring-like having an inner diameter 204 and an outer diameter 206. A tab 208 is disposed on the inner diameter 206 of the spacers 202.

The stem 112 includes groove 210 extending longitudinally along a length of the stem 112. The inner diameter 204 of the spacer 202, or spacers 202, is sized to slide over the stem 112. The tabs 208 are configured to slidingly fit into the groove 210 of the stem 112. The spacer 202 is configured to be assembled to the stem 112 utilizing the groove 210 of the stem 112 as a guide for the tab 208 of the spacer 202. The groove 210 of the stem 112 prevents rotation of the spacer 202 relative to the stem 112.

The implant 200 may include a plurality (or kit) 212 of spacers 202 that are configured to be assembled onto the stem 112. The kit 212 of spacers 202 advantageously provides a plurality of predetermined adjustable offsets 148 of the polymer head 102 from the bone 142 the implant 200 is implanted into. Therefore, much like the kit 156 of implants 100, the kit 212 of spacers 202 enables a surgeon to choose and adjust to the optimum offset 148 between the polymer head 102 and the distal end of the bone 142 during a surgical procedure. However, rather than picking the implant 100 with the correct spacer 122 and spacer height 126, the surgeon simply selects the number of spacers 202 required to slide over the stem 112 of implant 200 to acquire such an offset 148.

Additionally, the stem 112 of implant 200 may including a porous metal surface 214 that allows bone ingrowth to prevent rotation of the implant 200 within the bone 142 that the implant 200 is implanted into. By way of a non-limiting example, the porous metal surface 214 may be a porous metal wrap disposed on the stem 112 of the implant 200. The stem 112 of implant 200 may be press fit into the canal 144 of the bone 142 to fix the stem 112 within the bone. Therefore, this implant 200 may be designed not to spin.

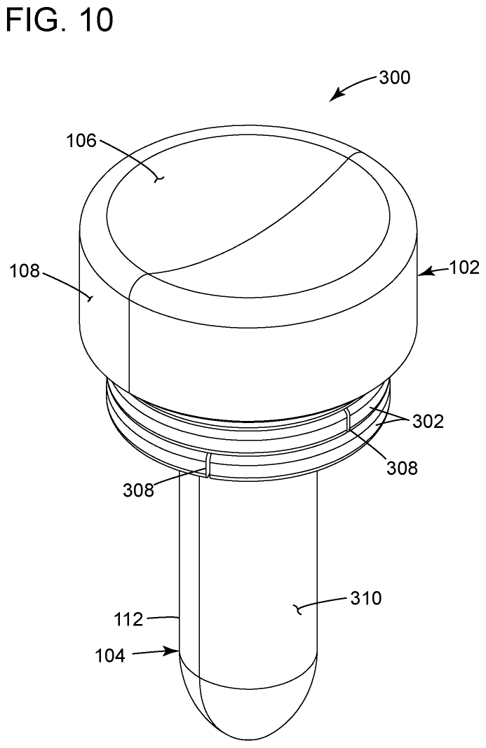

Referring to FIGS. 10 and 11, an example is depicted of a front top perspective view (FIG. 10) and a bottom perspective view (FIG. 11) of a third embodiment 300 of a radial head implant, in accordance with the present disclosure. The implant 300 is similar in many respects to that of implant 100 and, therefore, like or similar features are labeled with the same reference numbers.

Accordingly, implant 300 includes a polymer head 102 and a biocompatible metal stem component 104. The polymer head 102 includes an articulating surface 106 and an outer head perimeter 108. The stem component includes a stem 112 and a mounting platform 114 (see FIG. 3A). The stem 112 is configured to be implanted into a bone 142 of a patient. The mounting platform 114 is configured to have the polymer head 102 attached thereon. The outer head perimeter 108 of the polymer head 102 is larger than the mounting platform 114 outer perimeter 116. A first portion 118 of the polymer head 102 extends around the mounting platform outer perimeter 116 and under the mounting platform 114 to secure the polymer head 102 to the mounting platform 114 (see FIG. 4).

However, the spacers 302 of implant 300 are not integrally connected to the stem component 104. Rather the spacers 302 are configured as ring-like, having an inner diameter 304 and an outer diameter 306. A thru-slit 308 extends through one side of the spacer 302, from the outer diameter 306 to the inner diameter 304.

The stem 112 of implant 300 includes a smooth circular outer surface 310 that is configured to enable the implant 300 to spin within a canal 144 of the bone 142 that the implant 300 is implanted into. The stem has lower portion 312 having a first diameter 314 and an upper portion 316 having a second diameter 318 that is larger than the first diameter 316. The spacer's inner diameter 304 is larger than the first diameter 314 of the lower portion 312 of the stem 112 and smaller than the second diameter 318 of the upper portion 316 of the stem 112.

The spacer 302, or spacers 302, are configured to slide over the lower portion 312 of the stem 112 and to be press fit over the upper portion 316 of the stem 112. More specifically, the inner diameter 304 of the spacer 302 is sized larger than the first diameter 314 of the lower portion, so the spacer slides freely over the lower portion 312 of the stem 112 with substantially no interference. However, the inner diameter 304 of the spacer 302 is sized slightly smaller than the second diameter 318 so the upper portion 316 of the stem 112 presses against the inner perimeter of the spacer 302 to urge the thru-slit 308 of the spacer 302 to enlarge from a first slit width to a second larger slit width. Therefore, the spacer 302 is configured to be press fit over the upper portion 316 of the stem 112 and to secure itself to the upper portion 316 in an interference fit.

The implant 300 may include a plurality (or kit) 320 of spacers 302 that are configured to be assembled onto the stem 112. The kit 320 of spacers 302 advantageously provides a plurality of predetermined adjustable offsets 148 of the polymer head 102 from the bone 142 the implant 300 is implanted into. Therefore, much like the kit 212 of implants 200, the kit 320 of spacers 302 enables a surgeon to choose and adjust to the optimum offset 148 between the polymer head 102 and the distal end of the bone 142 during a surgical procedure. Also like kit 212 of implant 200, the surgeon simply selects the number of spacers 302 required to slide over the stem 112 of implant 300 to acquire such an offset 148.

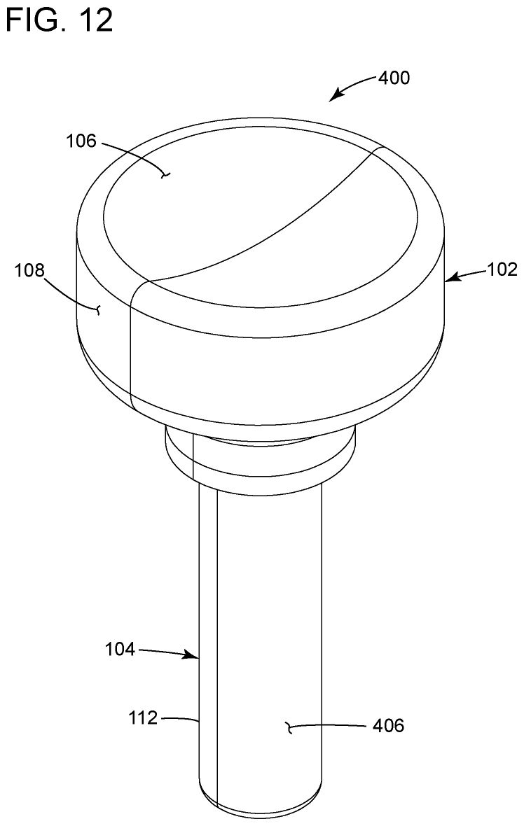

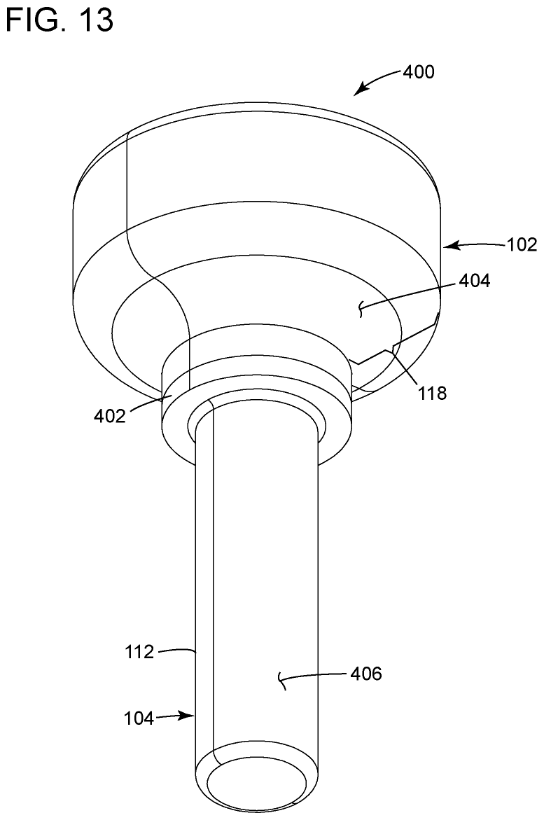

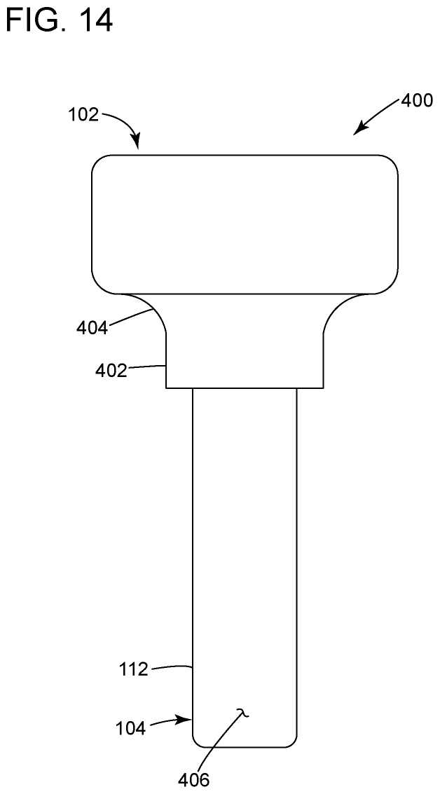

Referring to FIGS. 12, 13 and 14, an example is depicted of a front top perspective view (FIG. 12) and a bottom perspective view (FIG. 13) and a side view (FIG. 14) of a fourth embodiment 400 of a radial head implant, in accordance with the present disclosure. The implant 400 is similar in many respects to that of implant 100 and, therefore, like or similar features are labeled with the same reference numbers.

Accordingly, implant 400 includes a polymer head 102 and a biocompatible metal stem component 104. The polymer head 102 includes an articulating surface 106 and an outer head perimeter 108. The stem component includes a stem 112 and a mounting platform 114 (see FIG. 3A). The stem 112 is configured to be implanted into a bone 142 of a patient. The mounting platform 114 is configured to have the polymer head 102 attached thereon. The outer head perimeter 108 of the polymer head 102 is larger than the mounting platform 114 outer perimeter 116. A first portion 118 of the polymer head 102 extends around the mounting platform outer perimeter 116 and under the mounting platform 114 to secure the polymer head 102 to the mounting platform 114 (see FIG. 4).

The implant 400 has an anatomical design to better conform to the shape of the bone 142 the implant 400 is being implanted into. More specifically, the spacer 402 is integrally connected to the mounting platform 114 via a smooth curved surface 404 (best see FIG. 14), which provides an anatomical fit to the bone 142 the implant 400 is implanted into. The stem 112 includes a smooth circular outer surface 406 that is configured to enable the implant 400 to spin within the canal 144 of the bone 142 the implant 400 is implanted into.

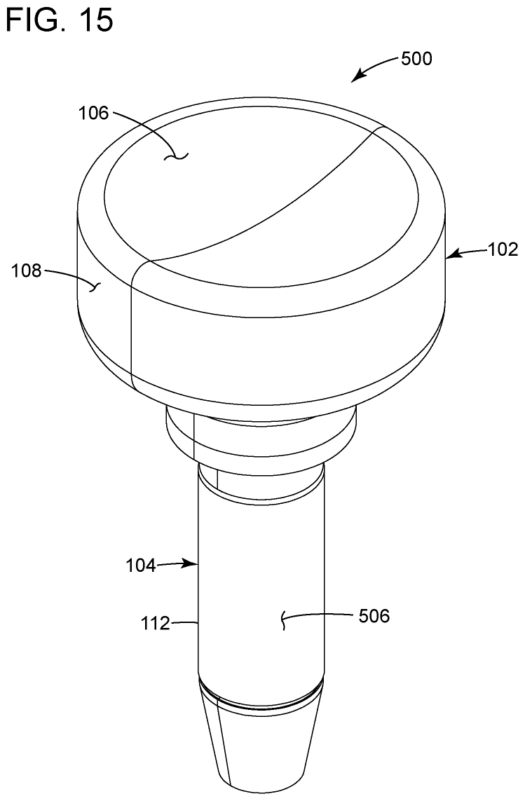

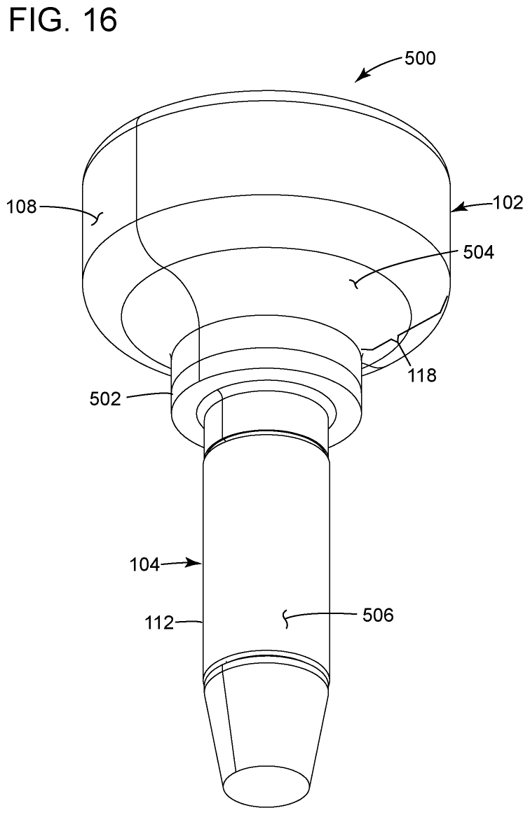

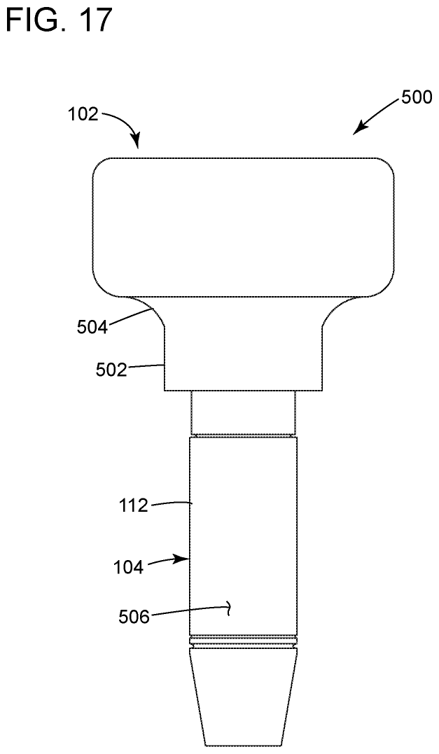

Referring to FIGS. 15, 16 and 17, an example is depicted of a front top perspective view (FIG. 15) and a bottom perspective view (FIG. 16) and a side view (FIG. 17) of a fifth embodiment 500 of a radial head implant, in accordance with the present disclosure. The implant 500 is similar in many respects to that of implants 100 and 400 and, therefore, like or similar features are labeled with the same reference numbers.

Accordingly, implant 400 includes a polymer head 102 and a biocompatible metal stem component 104. The polymer head 102 includes an articulating surface 106 and an outer head perimeter 108. The stem component includes a stem 112 and a mounting platform 114 (see FIG. 3A). The stem 112 is configured to be implanted into a bone 142 of a patient. The mounting platform 114 is configured to have the polymer head 102 attached thereon. The outer head perimeter 108 of the polymer head 102 is larger than the mounting platform 114 outer perimeter 116. A first portion 118 of the polymer head 102 extends around the mounting platform outer perimeter 116 and under the mounting platform 114 to secure the polymer head 102 to the mounting platform 114 (see FIG. 4).

The implant 500 has an anatomical design to better conform to the shape of the bone 142 the implant 500 is being implanted into. More specifically, the spacer 502 is integrally connected to the mounting platform 114 via a smooth curved surface 504 (best see FIG. 17), which provides an anatomical fit to the bone 142 the implant 500 is implanted into. The stem 112 of implant 500 may including a porous metal surface 506 that allows bone ingrowth to prevent rotation of the implant 500 within the bone 142 that the implant 500 is implanted into. By way of a non-limiting example, the porous metal surface 506 may be a porous metal wrap disposed on the stem 112 of the implant 500. The stem 112 of implant 500 may be press fit into the canal 144 of the bone 142 to fix the stem 112 within the bone. Therefore, this implant 500 may be designed not to spin.

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

Although the invention has been described by reference to specific examples, it should be understood that numerous changes may be made within the spirit and scope of the inventive concepts described. Accordingly, it is intended that the disclosure not be limited to the described examples, but that it has the full scope defined by the language of the following claims.

What is claimed is:

1. A radial head replacement implant, comprising:
a polymer head, comprising:
an articulating surface, and
an outer head perimeter; and
a biocompatible metal stem component comprising:
a stem, configured to be implanted into a bone of a patient, and
a mounting platform configured to have the polymer head attached thereon, the mounting platform having a mounting platform outer perimeter;
wherein the outer head perimeter of the polymer head is larger than the mounting platform outer perimeter, and wherein a first portion of the polymer head extends around the mounting platform outer perimeter and under the mounting platform to secure the polymer head to the mounting platform.

2. The implant of claim 1, comprising a spacer configured to be disposed on the stem and configured to contact the mounting platform, the spacer comprising:
a spacer diameter that is configured:
smaller than the mounting platform outer perimeter such that the spacer does not overlap the first portion of the polymer head that extends under the mounting platform, and
large enough to abut against an outer perimeter of the bone that the implant is implanted into; and
a spacer height that is configured to provide a predetermined offset of the polymer head from the bone the implant is implanted into.

3. The implant of claim 1, comprising:
the mounting platform comprising:
a circular upper platform having a first diameter that forms the mounting platform outer perimeter, and
a circular lower platform having a second diameter smaller than the first diameter;
wherein the second diameter of the circular lower platform is sized large enough to abut against an outer perimeter of the bone that the implant is implanted into;
wherein the upper platform and lower platform form a lip disposed on an under-surface of the upper platform between the first diameter and second diameter; and
wherein the first portion of the polymer head that extends under the mounting platform is disposed under the lip and is flush with an under-surface of the lower platform.

4. The implant of claim 1, comprising:
a first dovetail groove disposed in an upper surface of the mounting platform and extending across the mounting platform; and
wherein a second portion of the polymer head extends into the first dovetail groove to secure the polymer head to the mounting platform.

5. The implant of claim 4, comprising:
a second dovetail groove disposed in the upper surface of the mounting platform and extending perpendicularly relative to the first dovetail groove across the mounting platform;
wherein a third portion of the polymer head extends into the second dovetail groove to secure the polymer head to the mounting platform.

6. The implant of claim 2, comprising:
the spacer being integrally connected to the mounting platform and the stem; and
the stem comprising a smooth circular outer surface that is configured to enable the implant to spin within the bone the implant is implanted into.

7. The implant of claim 6, wherein the implant comprises a plurality of implants, each implant having a spacer with a different spacer height.

8. The implant of claim 2, comprising:
the stem having a groove extending longitudinally along a length of the stem;
the spacer having a tab configured to slidingly fit into the groove of the stem;
wherein the spacer is configured to be assembled to the stem utilizing the groove of the stem as a guide for the tab of the spacer; and
wherein the groove of the stem prevents rotation of the spacer relative to the stem.

9. The implant of claim 7, wherein spacer comprises:
a plurality of spacers configured to be assembled onto the stem, the plurality of spacers providing a plurality of predetermined offsets of the polymer head from the bone the implant is implanted into.

10. The implant of claim 8, comprising:
the stem including a porous metal surface that allows bone ingrowth to prevent rotation of the implant within the bone that the implant is implanted into.

11. The implant of claim 2, comprising:
the stem having a smooth lower portion having a first diameter and an upper portion having a second diameter that is larger than the first diameter;
the spacer including a thru-slit through one side of the spacer, and having an inner diameter that is larger than the first diameter of the stem and smaller than the second diameter of the stem;
wherein the spacer is configured to:
slide over the lower portion, and
be press fit over the upper portion, such that the second diameter of the upper portion presses against the inner diameter of the spacer to urge the thru-slit of the spacer to enlarge.

12. The implant of claim 11, wherein spacer comprises:
a plurality of spacers configured to be assembled onto the stem, the plurality of spacers providing a plurality of predetermined offsets of the polymer head from the bone the implant is implanted into.

13. The implant of claim 11, wherein the stem comprises a smooth circular outer surface that is configured to enable the implant to spin within the bone the implant is implanted into.

14. The implant of claim 2, comprising:
the spacer being integrally connected to the mounting platform via a smooth curved surface, which provides an anatomical fit to the bone the implant is implanted into; and
the stem comprising a smooth circular outer surface that is configured to enable the implant to spin within the bone the implant is implanted into.

15. The implant of claim 2, comprising:
the spacer being integrally connected to the mounting platform; and
the stem including a porous metal surface that allows bone ingrowth to prevent rotation of the implant within the bone that the implant is implanted into.

16. A radial head replacement implant, comprising:
a polymer head, comprising:
an articulating surface, and
an outer head perimeter; and
a biocompatible metal stem component comprising:
a stem, configured to be implanted into a bone of a patient,
a mounting platform configured to have the polymer head attached thereon, the mounting platform having a mounting platform outer perimeter; and
a spacer configured to be disposed on the stem and configured to contact the mounting platform, the spacer comprising:
a spacer diameter that is configured smaller than the mounting platform outer perimeter and large enough to abut against an outer perimeter of the bone that the implant is implanted into, and
a spacer height that is configured to provide a predetermined offset of the polymer head from the bone the implant is implanted into.

17. The implant of claim 16, wherein:
the outer head perimeter of the polymer head is larger than the mounting platform outer perimeter, and wherein a first portion of the polymer head extends around the mounting platform outer perimeter and under the mounting platform to secure the polymer head to the mounting platform; and
the spacer diameter is configured such that the spacer does not contact the first portion of the polymer head that extends under the mounting platform.

18. The implant of claim 16, wherein the mounting platform comprises:
a first dovetail groove disposed in an upper surface of the mounting platform and extending across the mounting platform, wherein a second portion of the polymer head extends into the first dovetail groove to secure the polymer head to the mounting platform.

19. The implant of claim 18, comprising:
a second dovetail groove disposed in the upper surface of the mounting platform and extending perpendicularly relative to the first dovetail groove across the mounting platform;
wherein a third portion of the polymer head extends into the second dovetail groove to secure the polymer head to the mounting platform.

20. The implant of claim 18, wherein the implant comprises a plurality of implants, each implant having a spacer with a different spacer height.

21. The implant of claim 16, comprising:
the spacer being integrally connected to the mounting platform and the stem; and
the stem comprising a smooth circular outer surface that is configured to enable the implant to spin within the bone the implant is implanted into.

22. The implant of claim 16, comprising:
the stem having a groove extending longitudinally along a length of the stem;
the spacer having a tab configured to slidingly fit into the groove of the stem;
wherein the spacer is configured to be assembled to the stem utilizing the groove of the stem as a guide for the tab of the spacer; and
wherein the groove of the stem prevents rotation of the spacer relative to the stem.

23. The implant of claim 16, comprising:
the stem having a smooth lower portion having a first diameter and an upper portion having a second diameter that is larger than the first diameter;
the spacer including a thru-slit through one side of the spacer, and having an inner diameter that is larger than the first diameter of the stem and smaller than the second diameter of the stem;
wherein the spacer is configured to:
slide over the lower portion, and
be press fit over the upper portion, such that the second diameter of the upper portion presses against the inner diameter of the spacer to urge the thru-slit of the spacer to enlarge.
